(19) United States
(12) Patent Application Publication
(10) Pub. No.: US 2022/0088199 A1
(43) Pub. Date: Mar. 24, 2022

(54) METHOD FOR PROTECTING GLYCEROL ALKYL ETHERS FROM OXIDATION

(71) Applicant: SACHEM, INC., Austin, TX (US)

(72) Inventor: Tim ENGEL, DR Tilburg (NL)

(21) Appl. No.: 17/520,934

(22) Filed: Nov. 8, 2021

Related U.S. Application Data
(63) Continuation of application No. 16/471,027, filed on Jun. 19, 2019, now abandoned.
(60) Provisional application No. 62/475,977, filed on Mar. 24, 2017.
(60) Provisional application No. 62/439,954, filed on Dec. 29, 2016.

(51) Int. Cl. A61K 47/10; A61K 8/34; A61K 8/49; A61K 47/22; A61Q 19/00

(52) U.S. Cl. CPC A61K 47/10; A61K 8/345; A61K 2800/522; A61K 47/22; A61Q 19/00; A61K 8/498

(57) ABSTRACT

Method of protecting glycerol alkyl ethers from oxidation, comprising steps (a.) and (b.):
a. providing a glycerol alkyl ether of the general formula (Ia) or (Ib):

R—O—CH₂—CHOH—CH₂OH (Ia) or

H—O—CH₂—CHOR—CH₂OH (Ib)
wherein, in general formula (Ia) or (Ib), R is C₃-C₁₈ branched or unbranched alkyl or alkenyl group, optionally substituted by a hydroxyl, C₁-C₄ alkoxy group, or both, and the C₃-C₁₈ alkyl group is optionally interrupted by oxygen atoms, or R is a C₆-C₁₀ aromatic hydrocarbon, optionally substituted by a hydroxyl, C₁-C₄ alkoxy group, or both, and
b. adding to the glycerol alkyl ether of step (a.) one or more compound selected from general formula (II):

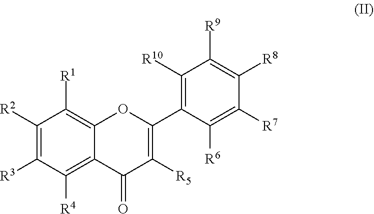

wherein each of R¹-R¹⁰ is hydrogen, hydroxyl, alkyl hydroxyl, alkoxy, alkyl ether, alkyl ester or glycoside, wherein the alkyl, alkoxy, and alkyl portion of the ester contains 1-4 carbon atoms, optionally branched, and the ester of the alkyl ester contains 1-5 carbon atoms, optionally branched.

### CROSS-REFERENCE TO RELATED APPLICATIONS

The present application is a continuation and claims priority under 35 U.S.C. § 120 to copending U.S. application Ser. No. 16/471,027, filed 19 Jun. 2019, now ______, which is a U.S. National Stage application which claims benefit under 35 U.S.C. 371 of International Application No. PCT/US2017/067780, which is based on and claims benefit under 35 U.S.C. § 119 of both U.S. Provisional Application No. 62/475,977, filed 24 Mar. 2017, and U.S. Provisional Application No. 62/439,954, filed 29 Dec. 2016, the entirety of each of which is hereby incorporated herein by reference.

### TECHNICAL FIELD

The present invention relates to compositions comprising glycerol monoalkyl ethers for use in cosmetic and pharmaceutical preparations and in technical products, and further comprising an anti-oxidant.

### BACKGROUND

Glycerol monoalkyl ethers are used as additives for cosmetic and pharmaceutical preparations. Glycerol monoalkyl ethers are used as physiologically compatible organic solvents. For example, 3-[(2-ethylhexyl)oxy]-1,2-propanediol has been used for some years as a deodorant active ingredient and skin care additive in cosmetic and pharmaceutical preparations. In such products, the glycerol monoalkyl ethers are added to the products in the form of a concentrate or, occasionally, as a pure glycerol monoalkyl ether.

During manufacture, storage and use, the glycerol monoalkyl ether, its concentrate and its dilute solution, which may be referred to as a working solution, are subject to stringent requirements for stability when used in personal care products, such as cosmetics, and pharmaceutical preparations. Because glycerol monoalkyl ethers occur naturally, both the naturally produced and the synthetically prepared members of the class of substance have become desirable for use in personal care products, such as cosmetics, and pharmaceutical preparations, and are widely accepted by manufacturers of cosmetics and pharmaceuticals as well as end users. It is known that glycerol monoalkyl ethers are subject to oxidative degradation, and so may be provided from the manufacturer with an antioxidant additive to inhibit such degradation. Over time, signs of instability, including formation of peroxide and formaldehyde, have been observed when the glycerol monoalkyl ethers are not stored properly.

Formation of peroxides and formaldehyde in cosmetic and pharmaceutical preparations can cause a number of problems. Formaldehyde is, in and of itself, highly undesirable in any exposure to humans or animals. In skin care products, the presence of peroxides can cause skin problems such as dermatosis. Peroxides can cause changes in the odor of stored products, due to oxidation of natural fats and oils present in such formulations. Peroxides may result in color changes, particularly in oil-in-water emulsions containing glycerol monoalkyl ethers. Peroxides can result in the formation of low molecular weight decomposition products that can be detected by chemical analysis, and in some cases, by smell. These problems resulting from peroxide formation in compositions containing glycerol monoalkyl ethers can result in rejection of products by quality control, or, worse, by customers and end users.

U.S. Pat. No. 6,956,062 discloses a number of antioxidants for use with glycerol monoalkyl ethers, including Vitamin E.

A drawback to using the antioxidants disclosed in U.S. Pat. No. 6,956,062 is that such antioxidants, as natural products, can be difficult to purify. Disadvantages of vitamin E may include the fact that sources and identity are not always clear, for example, whether the vitamin E material is synthetic or natural, the possibility that impurities, including unwanted color may be introduced, and the possibility that use of vitamin E may result in hypervitaminosis E from too much vitamin E being exposed to a user's body via a cosmetic or pharmaceutical composition containing same.

Therefore, a continuing need exists for new and improved antioxidants for use with glycerol monoalkyl ethers, particularly for use in personal care products, such as cosmetics, and pharmaceutical preparations.

### SUMMARY

The present inventors have discovered that a new class of antioxidant compounds provide particularly excellent results in preventing or at least reducing the incidence of the foregoing problems that may be encountered from the use of glycerol monoalkyl ethers in products such as cosmetics and pharmaceuticals. The new class of antioxidant compounds for use with glycerol monoalkyl ethers include compounds derived from flavanones, in particular, based on a 4H-1-Benzopyran-4-one, 2,3-dihydro-2-phenyl- or from flavones, based on a 4H-1-Benzopyran-4-one-2-phenyl-backbone, with a range of substitution at various locations on the base molecule.

Thus, in one embodiment, the present invention relates to a composition comprising:
a. a glycerol alkyl ether of the general formula (I):

R—O—CH₂—CHOH—CH₂OH (I)
wherein, in general formula (I), R is a C₃-C₁₈ alkyl group, in which the alkyl group is branched or unbranched, unsubstituted or substituted by one or more hydroxyl, one or more C₁-C₄ alkoxy groups, or both one or more hydroxyl and one or more C₁-C₄ alkoxy groups, and the C₃-C₁₈ alkyl group, whether branched or unbranched, unsubstituted or substituted, is optionally interrupted by up to four oxygen atoms, and
b. one or more compound selected from general formula (II):

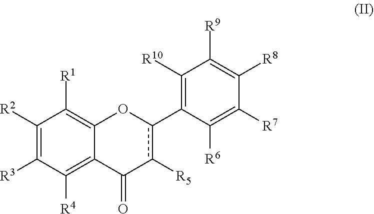

wherein, in the general formula (II), each of R¹-R¹⁰ is independently selected from hydrogen, hydroxyl, alkyl hydroxyl, alkoxy, alkyl ether, alkyl ester and O-glycoside, wherein, independently, the alkyl, the alkoxy and the alkyl portion of the alkyl ester contains from 1 to 4 carbon atoms, branched or unbranched, and the ester of the alkyl ester contains from 1 to 5 carbon atoms, branched or unbranched, the O-glycoside comprises a mono-, di-, or tri-saccharide, in each of which the saccharide is a natural or modified saccharide moiety bonded through an oxygen atom to the ring (thus, an O-glycoside); and wherein the dashed line indicates an optional carbon-carbon double bond. In some embodiments, when the optional carbon-carbon double bond is present, the resulting compound is a flavone. Similarly, in some embodiments, when the optional carbon-carbon double bond is not present, and instead a carbon-carbon single bond is present, the resulting compound is a flavanone.

In one embodiment of the composition of the present invention, the compound of general formula (II) is a flavone or a flavanone compound. In one embodiment, the compound of general formula (II) is a flavone or a flavanone compound either isolated from or known from a natural source, e.g., citrus or other fruit or a vegetable. In one embodiment the compound of formula (II) has the following general formula (IIa), a flavanone, or general formula (IIb), a flavone:

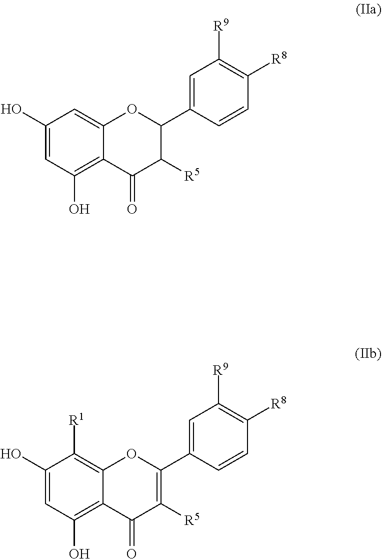

or a mixture of two or more compounds of the general formula (IIa) and general formula (IIb), in which the R¹, R⁵, R⁸ and R⁹ groups are as defined above for the compound of general formula (II).

In one embodiment of the composition of the present invention, the compound of general formula (II) is a flavonol compound. In one embodiment, the compound of general formula (II) is a flavonol compound either isolated from or known from a natural source, e.g., citrus or other fruit or a vegetable. In one embodiment the compound of formula (II) has the following general formula (IIc):

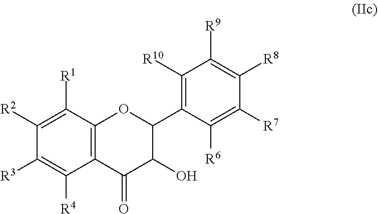

wherein, in the flavonol of general formula (IIc), the R groups have the definitions set forth above for the compound of general formula (II).

The foregoing compositions containing compounds of general formulae (I) and (II), and the method of preserving the compounds of general formula (I) for use in a cosmetic or pharmaceutical formulation, provides a novel and unexpectedly excellent method of providing a stable product containing the glycerol monoalkyl ether of general formula (I). The present invention thereby provides a solution to the long-standing problem of providing such a versatile and stable composition, and to the long-felt need for compositions that are particularly stable.

### BRIEF DESCRIPTION OF THE DRAWINGS

FIG. 1 is a graph showing stability test results comparing peroxide formation from both comparative and invention test samples.

FIG. 2 is a graph showing stability test results comparing formaldehyde formation from both comparative and invention test samples.

FIG. 3 is a graph showing the loss of 3-[(2-ethylhexyl)oxy]-1,2-propanediol on storage at elevated temperature with compounds in accordance with the present invention.

FIG. 4 is a graph showing the increase in volatile degradation products formed on storage of 3-[(2-ethylhexyl)oxy]-1,2-propanediol at elevated temperatures with compounds in accordance with the present invention.

FIG. 5 shows the results of stability testing of a blend of 3-[(n-octyl)oxy]-1,2-propanediol with a compound having general formula (II) in accordance with the present invention. FIG. 5a shows active assay and FIG. 5b shows degradation products.

FIG. 6 shows the results obtained when samples of SASKINE50™ are combined with a compound having general formula (II) in accordance with the present invention. FIG. 6a shows active assay and FIG. 6b shows degradation products.

FIG. 7 shows the minimum inhibitory concentrations for two of the glyceryl ethers with and without a compound having general formula (II) in accordance with the present invention.

FIG. 8 shows the results of skin irritation tests for 3-[(2-ethylhexyl)oxy]-1,2-propanediol (SASKINE50™) with and without a compound having general formula (II) in accordance with the present invention.

### DETAILED DESCRIPTION

As disclosed in the foregoing summary, the present invention relates to a composition containing a glycerol monoalkyl ether having a general formula (I) and an antioxidant having the general formula (II), in combination.

In one embodiment, the present invention relates to a composition comprising:
a. a glycerol alkyl ether of the general formula (I):

R—O—CH₂—CHOH—CH₂OH (I)

wherein, in general formula (I), R is a C₃-C₁₈ alkyl group, in which the alkyl group is branched or unbranched, unsubstituted or substituted by one or more hydroxyl, one or more C₁-C₄ alkoxy groups, or both one or more hydroxyl and one or more C₁-C₄ alkoxy groups, and the C₃-C₁₈ alkyl group, whether branched or unbranched, unsubstituted or substituted, is optionally interrupted by up to four oxygen atoms, and
b. one or more compound selected from general formula (II):

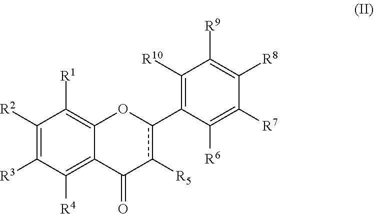

wherein, in the general formula (II), each of R¹-R¹⁰ is independently selected from hydrogen, hydroxyl, alkyl hydroxyl, alkoxy, alkyl ether, alkyl ester and O-glycoside, wherein, independently, the alkyl, the alkoxy and the alkyl portion of the alkyl ester contains from 1 to 4 carbon atoms, branched or unbranched, and the ester of the alkyl ester contains from 1 to 5 carbon atoms, branched or unbranched, the O-glycoside comprises a mono-, di-, or tri-saccharide, in each of which the saccharide is a natural or modified saccharide moiety bonded through an oxygen atom to the ring (thus, an O-glycoside); and wherein the dashed line indicates an optional carbon-carbon double bond. In some embodiments, the compound of general formula (II) is a flavonoid. In some embodiments, when the optional carbon-carbon double bond is present, the resulting flavonoid is a flavone or a flavonol. Similarly, in some embodiments, when the optional carbon-carbon double bond is not present, and instead a carbon-carbon single bond is present, the resulting flavonoid is a flavanone.

In one embodiment, the compound of general formula (II) comprises one or a mixture of two or more of compounds having the general formulae (IIa), (IIb), or (IIc):

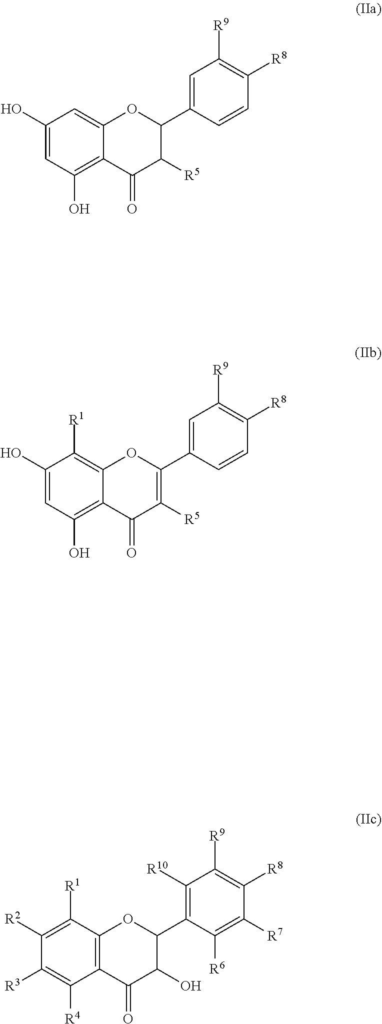

wherein, in the general formulae (IIa), (IIb), and (IIc), each of R¹-R¹⁰ are as defined above for the compound of general formula (II).

While the compounds of general formula (I) in embodiments of the present invention generally relate to glycerol monoalkyl ethers, i.e., to both the 2-substituted glycerol monoalkyl ether and the 3-substituted glycerol monoalkyl ether, the present invention relates in particular to the 3-substituted glycerol monoalkyl ether compounds.

It is noted that in the compounds according to general formula (I), the 2-position carbon atom of the glycerol moiety is a non-symmetric carbon atom. Accordingly, the glycerol monoalkyl ethers according to the invention can be present as racemic mixture (D,L) or in the form of enantiomer-enriched mixtures of the D- or L-form, or in the form of the pure D-enantiomer or the pure L-enantiomer. As used herein, unless specifically directed to one enantiomer or the other, there is no differentiation between the D- or L-enantiomers.

In one embodiment, the alkyl group in the glycerol monoalkyl ether is a C₃-C₁₂ hydrocarbon group, which may be branched or unbranched. In one embodiment, the alkyl in the glycerol monoalkyl ether group is a C₄-C₁₂ hydrocarbon group, branched or unbranched. In one embodiment, the alkyl group in the glycerol monoalkyl ether is a C₆-C₁₀ hydrocarbon group, branched or unbranched. In one embodiment, the alkyl group in the glycerol monoalkyl ether is a C₈ hydrocarbon group, branched or unbranched. In one embodiment, the C₈ hydrocarbon group is n-octyl; in one embodiment, the C₈ hydrocarbon group is 2-octyl; and in one embodiment, the C₈ hydrocarbon group is 2-ethylhexyl.

In one embodiment, the alkyl chain in the glycerol monoalkyl ether, whether branched or unbranched, is interrupted by up to 4 oxygen atoms. Thus, the alkyl chain in the alkyl group R of the glycerol monoalkyl ether can contain alkyleneoxy groups, such as, for example, ethyleneoxy and/or propyleneoxy groups. In this embodiment, the ether-containing moiety may be obtained, for example, by the reaction of an alcohol or a diol with ethylene oxide and/or propylene oxide, as appropriate depending on the length of the ether-containing moiety and the number of interrupting oxygen atoms. In another embodiment, the ether containing moiety may be obtained by hydrolysis of an alkyl glycidyl ether. in which the alkyl group may be branched or unbranched. Suitable ether-containing moieties for the R group include CH₃CH₂(OCH₂CH₂)ₙ—, in which n=1-5, CH₃CH₂(OCH(CH₃)CH₂)ₙ—, in which n=1-3, for example.

In one embodiment, the R alkyl moiety contains 4 to 12 carbon atoms, in one embodiment, from 6 to 10 carbon atoms, and in one embodiment, 8 carbon atoms. In one embodiment, the 8 carbon atom alkyl group is a 2-ethylhexyl group, and in another embodiment, the 8 carbon atom alkyl group is n-octyl. In one embodiment, glycerol monoalkyl ether is 3-[(2-ethylhexyl)oxy]-1,2-propanediol, which is marketed under the trade name SASKINE50™ by SACHEM, Inc., Austin, Tex.

In one embodiment, the branched or unbranched alkyl group includes hydroxyl or alkoxyl substitution along the length of the alkyl group. When the substitution is alkoxy, the alkyl portion of the alkoxy group is a C₁-C₄ alkyl moiety. When the substitution is alkoxy, each such substituent independently may be —OCH₃, —OCH₂CH₃, —OCH₂CH₂CH₃, —OCH(CH₃)₂, —OCH₂CH₂CH₂CH₃, —OCH₂CH(CH₃)₂, —OCH(CH₃)CH₂CH₃ or —OC(CH₃)₃. There may be multiple alkoxy groups.

In one embodiment, the branched or unbranched alkyl group includes alkyl hydroxyl substitution, in which the alkyl hydroxyl includes a C₁-C₄ alkyl moiety. When the substitution is alkyl hydroxyl, each such substituent independently may be —CH₂OH, —CH₂CH₂OH, —CH₂CH₂CH₂OH, —CH(CH₃)CH₂OH, —CH₂CH₂CH₂CH₂OH, —CH(CH₃)CH₂CH₂OH, —CH₂CH(CH₃)CH₂OH, or —CH₂C(CH₃)₂OH. There may be multiple alkyl hydroxyl groups.

In one embodiment of the composition of the present invention, in the general formula (I), R is a branched or unbranched C₄-C₁₂ alkyl group. In one embodiment, in the general formula (I), R is a branched or unbranched C₆-C₁₀ alkyl group. In one embodiment, in the general formula (I), R is a branched or unbranched C₈ alkyl group.

In one embodiment of the composition of the present invention, in the general formula (I), R is 2-ethylhexyl.

In one embodiment of the composition of the present invention, in the general formula (I), R is n-octyl.

In one embodiment of the composition of the present invention, in the general formula (I), R is 2-octyl.

In one embodiment of the composition of the present invention, the compound of general formula (II) is a flavone or a flavanone compound. In one embodiment, the compound of general formula (II) is a flavone or a flavanone compound either isolated from or known from a citrus fruit. In one embodiment the compound of formula (II) has the following general formula (IIa), a flavanone, or general formula (IIb), a flavone:

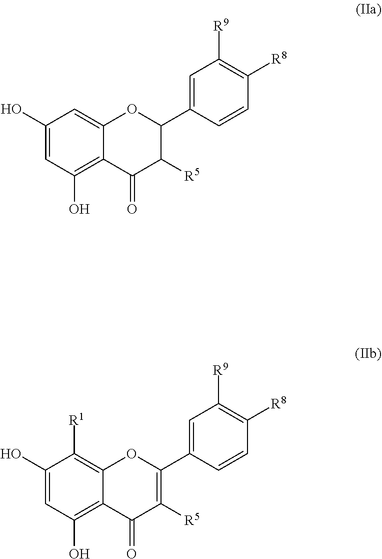

or a mixture of two or more compounds of the general formula (IIa) and general formula (IIb).

In one embodiment of the composition of the present invention, the compound of general formula (IIa) is one of the following flavanones, wherein in the general formula (II) above, the 2,3 carbon-carbon double bond is not present, and R¹, R³, R⁶, R⁷ and R¹⁰═H:

| Compound Name | R⁵ | R⁹ | R⁸ |
|---|---|---|---|
| Hesperetin | H | OH | OCH₃ |
| Naringenin | H | H | OH |
| Taxifolin (Epicatechin) | OH | OH | OH |
| Isokuranetin | H | H | OCH₃ |
| Eriodictyol | H | OH | OH |
| Aromadendrin (Aromadedrin) | OH | H | OH |

It is noted that, in the foregoing compounds according to general formula (IIa), in the pyran ring, the carbon atom bonded to the phenyl ring is asymmetric, and the carbon atom bonded to R⁵ is asymmetric when R⁵═OH. For example, taxifolin and epicatechin are diastereomers, since each have an OH group at R⁵ but in taxifolin the OH group at R⁵ is trans to the phenyl group, while in epicatechin, the OH group at R⁵ is cis to the phenyl group. The same applies to aromadendrin and aromadedrin, respectively, which are also diastereomers.

In one embodiment of the composition of the present invention, the compound of general formula (IIb) is one of the following flavones, wherein in the general formula (II) above, the 2,3 carbon-carbon double bond is present, and R³, R⁶, R⁷ and R¹⁰═H:

| Compound Name | R⁵ | R¹ | R⁹ | R⁸ |
|---|---|---|---|---|
| Acacetin | H | H | H | OCH₃ |
| Isocutellarein | H | OH | H | OH |
| Luteolin | H | H | OH | OH |
| Kaempferol | OH | H | H | OH |
| Quercetin | OH | H | OH | OH |
| Apigenin | H | H | H | OH |
| Diosmetin | H | H | OH | OCH₃ |
| Chrysoeriol | H | H | OCH₃ | OH |
| Chrysin | H | H | H | H |
| Galangin | OH | H | H | H |
| Limocitrin | OH | OCH₃ | OCH₃ | OH |

In one embodiment of the composition of the present invention, the compound having the general formula (II) is one or a mixture of two or more compounds selected from hesperetin, naringenin, taxifolin, epicatechin, isokuranetin, eriodictyol, aromadendrin, aromadedrin, acacetin, isocutellarein, luteolin, kaempferol, quercetin, apigenin, diosmetin, chrysoeriol, chrysin, and galangin. In one embodiment of the composition of the present invention, the compound having the general formula (II) is one or a mixture of two or more compounds selected from the group consisting essentially of hesperetin, naringenin, taxifolin, epicatechin, isokuranetin, eriodictyol, aromadendrin, aromadedrin, acacetin, isocutellarein, luteolin, kaempferol, quercetin, apigenin, diosmetin, chrysoeriol, chrysin, and galangin. Here, “consisting essentially of” means that no other flavonoid would be present in the compositions. In one embodiment of the composition of the present invention, the compound having the general formula (II) is one or a mixture of two or more compounds selected from the group consisting of hesperetin, naringenin, taxifolin, epicatechin, isokuranetin, eriodictyol, aromadendrin, aromadedrin, acacetin, isocutellarein, luteolin, kaempferol, quercetin, apigenin, diosmetin, chrysoeriol, chrysin, and galangin.

In one embodiment of the composition of the present invention, the compound of general formula (II) is a flavonol compound. In one embodiment, the compound of general formula (II) is a flavonol compound either isolated from or known from a natural source, e.g., citrus or other fruit or a vegetable. In one embodiment the compound of formula (II) has the following general formula (IIc):

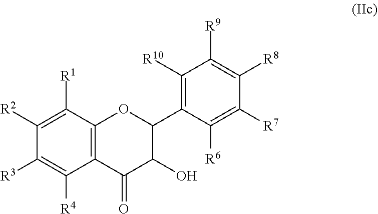

wherein, in the flavonol of general formula (IIc), the R groups have the definitions set forth above for the compound of general formula (II). In one embodiment of the composition of the present invention, the compound of general formula (IIc) is one of the following flavonols, wherein in the general formula (II) above, the 2,3 carbon-carbon double bond is absent, and R⁵═OH, R⁶═H, and the other R groups are as shown here:

| Name | R⁴ | R³ | R² | R¹ | R¹⁰ | R⁹ | R⁸ | R⁷ |
|---|---|---|---|---|---|---|---|---|
| Azaleatin | OCH₃ | H | OH | H | H | H | OH | OH |
| Fisetin | H | H | OH | H | H | OH | OH | H |
| Gossypetin | OH | H | OH | OH | H | OH | OH | H |
| Kaempferide | OH | H | OH | H | H | H | OCH₃ | H |
| Isorhamnetin | OH | H | OH | H | H | OCH₃ | OH | H |
| Morin | OH | H | OH | H | OH | H | OH | H |
| Myricetin | OH | H | OH | H | H | OH | OH | OH |
| Natsudaidain | OCH₃ | OCH₃ | OCH₃ | OCH₃ | H | H | OCH₃ | OCH₃ |
| Pachypodol | OH | H | OCH₃ | H | H | OCH₃ | OH | H |
| Quercetin | OH | H | OH | H | H | OH | OH | H |
| Rhamnazin | OH | H | OCH₃ | H | H | OCH₃ | OH | H |
| Rhamnetin | OH | H | OCH₃ | H | H | OH | OH | H |

In one embodiment, one or more of the R groups in general formula (IIc) is a sugar moiety, which forms a glycoside from the flavonol. Examples of flavonol glycosides include the following:

| Glycoside | Aglycone | R⁵ | R² | R⁸ |
|---|---|---|---|---|
| Astragalin | Kaempferol | Glucose | | |
| Azalein | Azaleatin | Rhamnose | | |
| Hyperoside | Quercetin | Galactose | | |
| Isoquercitin | Quercetin | Glucose | | |
| Kaempferitrin | Kaempfero | Rhamnose | Rhamnose | |
| Myricitrin | Myricetin | Rhamnose | | |
| Quercitrin | Quercetin | Rhamnose | | |
| Robinin | Kaempferol | Robinose | Rhamnose | |
| Rutin | Quercetin | Rutinose | | |
| Spiraeoside | Quercetin | | | Glucose |
| Xanthorhamnin | Rhamnetin | trisaccharide | | |

It is noted that, in the compounds shown above according to general formula (IIa), when R⁵═OH, the resulting compound is a flavonol. The foregoing classifications as a flavanone or a flavonol are for ease of reference only, and are not intended to be limiting or to exclude other compounds that fall within the definitions of the general formula (II) above.

A number of the compounds of general formulae (IIa), (IIb) and (IIc) can be obtained from a variety of natural sources, including both fruits and vegetables. One such source is citrus, e.g., orange peel or grapefruit peel or the fruit itself. It is well known that citrus generally, and citrus fruit and peel specifically, provides many healthful benefits. Additional natural sources of compounds in accordance with the general formula (II) include, but are not limited to the following examples: black tea, beer, apples, bananas, blueberries, peaches, pears, strawberries, oranges, grapefruit, lemons, tomatoes, tangerines, tangelos, parsley, pepper, celery, watermelon, lettuce, cherries, cabbage, cranberries, plums, raspberries, black beans, and onions. Compounds in accordance with general formula (II) obtained from such sources are natural products that can be incorporated into a cosmetic or pharmaceutical product. Similar synthetic and chemically modified compounds falling within the scope of the general formula (II) are also within the scope of the present invention.

In one embodiment of the composition of the present invention, in the one or more compound selected from the general formula (II), each of R¹-R¹⁰ is independently selected from hydrogen, hydroxyl, alkyl hydroxyl and alkoxy.

In one embodiment of the composition of the present invention, in the general formula (I), R is a branched or unbranched C₄-C₁₂ alkyl group and in the one or more compound selected from the general formula (II), each of R¹-R¹⁰ is independently selected from hydrogen, hydroxyl, C₁-C₄ alkyl hydroxyl and C₁-C₄ alkoxy.

In one embodiment of the composition of the present invention, the one or more compound selected from the general formula (II), one or more of the R groups is an O-glycoside; in some embodiments the O-glycoside replaces the OH group at R² in the compounds of general formulae (IIa) or (IIb). In one embodiment, the glycoside is rutinose, and in another embodiment, the glycoside is neohesperidose. In another embodiment, the glycoside is rhamnose. In another embodiment, the glycoside is glucose. In another embodiment, the glycoside is xylose.

In one embodiment, when the O-glycoside is rutinose, the flavanone is eriocitrin, obtained when the R² hydroxyl group of eriodictyol is replaced by O-rutinose. In one embodiment, when the O-glycoside is rutinose, the flavanone is narirutin, obtained when the R² hydroxyl group of apigenin is replaced by O-rutinose. In one embodiment, when the O-glycoside is rutinose, the flavanone is hesperiden, obtained when the R² hydroxyl group of hesperitin is replaced by O-rutinose. In one embodiment, when the O-glycoside is rutinose, the flavanone is neoponcirin, obtained when the R² hydroxyl group of isosakuranetin is replaced by O-rutinose.

In one embodiment, when the O-glycoside is neohesperidose, the flavanone is neoeriocitrin, obtained when the R² hydroxyl group of eriodictyol is replaced by O-neohesperidose. In one embodiment, when the O-glycoside is neohesperidose, the flavanone is naringin, obtained when the R² hydroxyl group of apigenin is replaced by O-neohesperidose. In one embodiment, when the O-glycoside is neohesperidose, the flavanone is neohesperiden, obtained when the R² hydroxyl group of hesperitin is replaced by O-neohesperidose. In one embodiment, when the O-glycoside is neohesperidose, the flavanone is poncirin, obtained when the R² hydroxyl group of isosakuranetin is replaced by O-neohesperidose.

In one embodiment, when the O-glycoside is rutinose, the flavone is rutin, obtained when the R² hydroxyl group of quercitin is replaced by O-rutinose. In one embodiment, when the O-glycoside is rutinose, the flavone is isorhoifolin, obtained when the R² hydroxyl group of apigenin is replaced by O-rutinose. In one embodiment, when the O-glycoside is rutinose, the flavone is deosmin, obtained when the R² hydroxyl group of diosmetin is replaced by O-rutinose.

In one embodiment, when the O-glycoside is neohesperidose, the flavone is rhoifolin, obtained when the R² hydroxyl group of apigenin is replaced by O-neohesperidose. In one embodiment, when the O-glycoside is neohesperidose, the flavone is neodeosmin, obtained when the R² hydroxyl group of diosmetin is replaced by O-neohesperidose.

In one embodiment, independently, the foregoing glycosidic flavanones and flavones are known, obtained, or both known and obtained, from citrus. In one embodiment, independently, the foregoing glycosidic flavanones and flavones are known, obtained, or both known and obtained, from black tea, beer, apples, bananas, blueberries, peaches, pears, strawberries, oranges, grapefruit, lemons, tomatoes, tangerines, tangelos, parsley, pepper, celery, watermelon, lettuce, cherries, cabbage, cranberries, plums, raspberries, black beans, and onions.

In one embodiment of the composition of the present invention, in the one or more compound selected from the general formula (II), R¹, R³, R⁶, R⁷ and R¹⁰ are H, R² and R⁴ are OH, R⁵ and R⁹ are H or OH, and R⁸ is OH or C₁-C₄ alkoxy. Thus, for example and in accordance with certain ones of this embodiment, the compound selected from general formula (II) has one of the following structures (IIe), (IIf), (IIg) or (IIh):

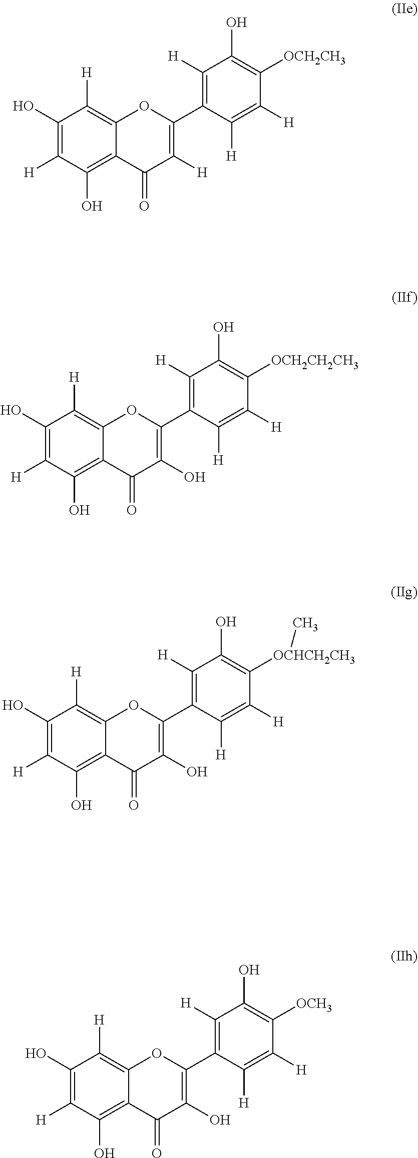

As in general formula (II), in (IIe-IIh), the double bond in the pyran ring is optional.

In one embodiment of the composition of the present invention, the one or more compound selected from the general formula (II) is one or a combination of two or more of naringenin, quercetin, hesperetin and eriodictyol, as shown here:

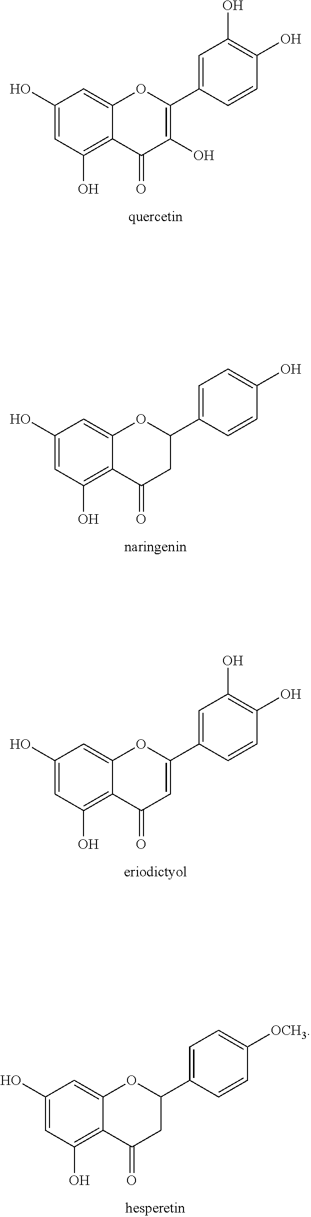

In one embodiment of the composition of the present invention, in the compound selected from the general formula (I), R is 2-ethylhexyl or R is n-octyl and the one or more compound selected from the general formula (II), is one or a combination of two or more of naringenin, quercetin, hesperetin and eriodictyol, as shown above.

In one embodiment of the composition of the present invention, R is 2-ethylhexyl or R is n-octyl and the one or more compound selected from the general formula (II), is one or a combination of two or more of hesperetin, naringenin, taxifolin, epicatechin, isokuranetin, eriodictyol, aromadendrin, aromadedrin, acacetin, isocutellarein, luteolin, kaempferol, quercetin, apigenin, diosmetin, chrysoeriol, chrysin, and galangin.

In one embodiment of the composition of the present invention, the compound having the general formula (II) is a polymethoxylated flavone, i.e., a polymethoxylated compound having a general formula (IIb). In one embodiment, the compound having a general formula (II), as defined above, includes methoxy groups at each of R², R³ and R⁸, H atoms at R⁶, R⁷ and R¹⁰, and, includes additional methoxy groups at one or more of R¹, R⁴, R⁵, and R⁹ to form the following compounds:

| Name | R¹ | R⁴ | R⁵ | R⁹ |
|---|---|---|---|---|
| Sinensetin | H | OCH₃ | H | OCH₃ |
| Nobiletin | OCH₃ | OCH₃ | H | OCH₃ |
| Heptamethoxyflavone | OCH₃ | OCH₃ | OCH₃ | OCH₃ |
| Natsudaidain | OCH₃ | OCH₃ | OH | OCH₃ |
| 5-demethylnobiletin | OCH₃ | OH | H | OCH₃ |
| Tangeretin | OCH₃ | OCH₃ | H | H |

Other known polymethoxylated flavones include tetra-O-methylscutellarein, tetra-O-methylisoscutellarein, hexa-O-methylquercetagetin, hexa-O-methylgossypetin, and 5-Hydroxy-3,7,8,3′,4′-pentamethoxyflavone.

In one embodiment of the composition of the present invention, in the compound selected from the general formula (I), R is 2-ethylhexyl or R is n-octyl and the one or more compound selected from the general formula (II), is one or a combination of two or more of naringenin, quercetin, hesperetin and eriodictyol, as shown above.

In one embodiment of the composition of the present invention, in the general formula (I), R is a branched or unbranched C₄-C₁₂ alkyl group and in the one or more compound selected from the general formula (II), R¹, R³, R⁶, R⁷ and R¹⁰ are H, R² and R⁴ are OH, R⁵ and R⁹ are H or OH, and R⁸ is OH or C₁-C₄ alkoxy.

In one embodiment of the composition of the present invention, in the general formula (I), R is a branched or unbranched C₄-C₁₂ alkyl group and in the one or more compound selected from the general formula (II), each of R¹-R¹⁰ is independently selected from hydrogen, hydroxyl, C₁-C₄ alkyl hydroxyl and C₁-C₄ alkoxy.

In one embodiment of the composition of the present invention, in the general formula (I), R is a branched or unbranched C₄-C₁₂ alkyl group and in the one or more compound selected from the general formula (II), R¹, R³, R⁶, R⁷ and R¹⁰ are H, R² and R⁴ are OH, R⁵ and R⁹ are H or OH, and R⁸ is OH or C₁-C₄ alkoxy.

In one embodiment of the composition of the present invention, in the general formula (I), R is 2-ethylhexyl or R is n-octyl and in the one or more compound selected from the general formula (II), each of R¹-R¹⁰ is independently selected from hydrogen, hydroxyl, C₁-C₄ alkyl hydroxyl and C₁-C₄ alkoxy.

In one embodiment of the composition of the present invention, in the general formula (I), R is 2-ethylhexyl or R is n-octyl and in the one or more compound selected from the general formula (II), R¹, R³, R⁶, R⁷ and R¹⁰ are H, R² and R⁴ are OH, R⁵ and R⁹ are H or OH, and R⁸ is OH or C₁-C₄ alkoxy.

Specific combinations in accordance with select embodiments of the present invention include, but are not limited to:
3-[(2-ethylhexyl)oxy]-1,2-propanediol and naringenin;
3-[(2-ethylhexyl)oxy]-1,2-propanediol quercetin;
3-[(2-ethylhexyl)oxy]-1,2-propanediol and hesperetin;
3-[(2-ethylhexyl)oxy]-1,2-propanediol and eriodyctiol;
3-[(n-octyl)oxy]-1,2-propanediol and naringenin;
3-[(n-octyl)oxy]-1,2-propanediol quercetin;
3-[(n-octyl)oxy]-1,2-propanediol and hesperetin;
3-[(n-octyl)oxy]-1,2-propanediol and eriodyctiol;
3-[(2-octyl)oxy]-1,2-propanediol and naringenin;
3-[(2-octyl)oxy]-1,2-propanediol quercetin;
3-[(2-octyl)oxy]-1,2-propanediol and hesperetin;
3-[(2-octyl)oxy]-1,2-propanediol and eriodyctiol.

In one embodiment, the compound having the general formula (II) is one or more selected from Acacetin, Amurensin, Apigenin, Apigetrin, Azalein, Azaleatin, Baicalein, Butin, Chrysin, Chrysoeriol, Diosmin, Diosmetin, Eriocitrin, Eriodictyol, Eupafolin, Eupatilin, Fisetin, Flavoxate, Galangin, Genkwanin, Gossypetin, Hesperetin, Hispidulin, Homoeriodictyol, Hyperoside, Icariin, Isosakuranetin, Isoquercitin, Isorhamnetin, Kaempferide, Kaempferitrin, Kaempferol, Luteolin, Likvirtin, Liquiritin, Liquiritigenin, Morin, Myricetin, Myricitrin, Naringenin, Naringin, Natsudaidain, Neohesperidin, Nobiletin, Pachypodol, Pinocembrin, Poncirin, Quercetin, Quercitrin, Rhamnetin, Rhamnazin, Rhoifolin, Robinin, Robinose, Rutinose, Sakuranetin, Sakuranin, Scutellarein, Spiraeoside, Spirenoside, Sterubin, Tangeretin, Tangeritin, Techtochrysin, Troxerutin, Wogonin, and Xanthorhamnin.

In one embodiment of the composition of the present invention, the composition contains from about 50 ppm to about 50000 ppm (equivalent to 0.005 wt % to 5 wt %) of the compound of general formula (II), based on the content (weight) of the glycerol monoalkyl ether. Thus, using this embodiment as an example, to a concentrate containing otherwise substantially pure glycerol monoalkyl ether, there will be added from about 50 ppm to about 50000 ppm of the antioxidant of general formula (II). In one embodiment, the composition contains from about 100 ppm to about 10000 ppm of the compound of general formula (II), based on the content of the glycerol monoalkyl ether. In one embodiment, the composition contains from about 200 ppm to about 5000 ppm of the compound of general formula (II), based on the content of the glycerol monoalkyl ether. In one embodiment, the composition contains from about 500 ppm to about 1000 ppm of the compound of general formula (II), based on the content of the glycerol monoalkyl ether. In one embodiment, the composition contains from about 300 ppm to about 900 ppm of the compound of general formula (II), based on the content of the glycerol monoalkyl ether.

When a composition in accordance with embodiments of the present invention is used in a cosmetic or pharmaceutical formulation, it will be added and the foregoing ratio will be maintained, between the compound of general formula (I) and the compound of general formula (II). Optionally, it may be desirable to add additional quantities of the compound of general formula (II) to the cosmetic or pharmaceutical formulation, as additional antioxidant for the cosmetic or pharmaceutical composition.

In one embodiment of the composition of the present invention, the composition is a concentrate containing from 0.01 wt % to 5 wt % of the of the compound of the general formula (II), based on the total weight of the composition, with the remainder of the concentrate being one or more compound of the general formula (I). In one embodiment, the concentrate may also include other ingredients, and the concentrate will still contain from 0.01 wt % to 5 wt % of the compound of general formula (II), based on the total weight of the compound of general formula (I) present in the concentrate.

In one embodiment of the composition of the present invention, the compound of general formula (I) is provided to the composition at a purity of at least 99.99%, prior to addition of the compound of general formula (II). In one embodiment, the compound of general formula (I) is provided to the composition at a purity of at least 99%, prior to addition of the compound of general formula (II). In one embodiment, the compound of general formula (I) is provided to the composition at a purity of at least 98%, prior to addition of the compound of general formula (II). In one embodiment, the compound of general formula (I) is provided to the composition at a purity of at least 94%, prior to addition of the compound of general formula (II). These levels of purity of the compound of general formula (I) ensure that the product contains few if any impurities that might be absorbed through the skin of a person using a cosmetic or pharmaceutical composition made using the composition of the present invention.

In one embodiment, the composition according to the invention can be provided in the form of a working solution. A working solution may comprise from 10% by weight to about 60% by weight of the composition, including both the one or more compound according to general formula (I) and the one or more compound according to general formula (II), as defined herein. To obtain such a working solution, a concentrate containing the components (a) and (b) according to the invention can be dissolved in, i.e., diluted by, a suitable amount of an additive, such as, for example, water, alcohol(s) or polyol(s), or mixtures of water, alcohol(s) and/or polyol(s). In such working solutions, it is considered that the relative amounts of the one or more compound according to general formula (I) and of the one or more compound according to general formula (II) disclosed above for the concentrates according to the invention, remain the same when the working solution is prepared from the concentrate by dilution. As noted above, optionally, additional quantities of the one or more compound according to general formula (II) may be added.

The compositions according to embodiments of the invention, either in the form of a concentrate or as a working solution, can be added to cosmetic and/or pharmaceutical preparations, for example, as is known from the use of glycerol monoalkyl ethers. In other embodiments, compositions according to embodiments of the invention may be used in technical products which are intended to be provided with glycerol monoalkyl ethers and in which peroxides are undesired, e.g., preparations comprising compounds which contain dyes or perfumes or which are unsaturated or sensitive to oxidation. Such preparations or technical products may include, for example, deodorants, skincare products, sunscreens, baby products, cosmetics, aftershaves, disinfectants, antiseptics, washing lotions, hair treatment compositions.

In some embodiments, the compositions according to the invention, whether added as concentrates or working solutions, are used in the preparations such as those examples above, such that the corresponding cosmetic, pharmaceutical or technical preparation contains from about 0.05 to about 5 wt %, or in another embodiment, from about 0.1 to about 1 wt %, or in another embodiment, from about 0.2 to about 0.6 wt %, or, in other embodiments 0.3 wt % or 0.5%, of the glycerol monoalkyl ether of the general formula (I) as defined herein. It is noted that, as the purity of the compound of formula (I) is increased, e.g., from 94% pure up to 98% pure, prior to distillation, a somewhat higher level of the antioxidant compound according to general formula (II) may be required, since it appears that higher purity compound according to general formula (I) needs greater stabilization.

The foregoing example products may be prepared by simple mixing of the composition of the present invention together with the other components of the products.

### EXAMPLES

### Example 1

To demonstrate the antioxidant capabilities of the present invention, mixtures of 3-[(2-ethylhexyl)oxy]-1,2-propanediol, a compound having a general formula (I) as defined herein, which is marketed under the trade name SASKINE50™ by SACHEM, Inc., Austin, Tex., with naringenin, one of the compounds of general formula (II) as defined herein, are prepared. The thus-prepared compositions are subjected to stability testing, under which formation of formaldehyde and peroxides and determined as indicators of stability, in which higher amounts of formaldehyde and/or peroxides are deemed to show lower stability of the mixture. As a first comparative example, a sample of 3-[(2-ethylhexyl)oxy]-1,2-propanediol, a compound having a general formula (I) as defined herein, is tested with no added antioxidant. As a second comparative example, a sample of commercially available 3-[(2-ethylhexyl)oxy]-1,2-propanediol, a compound having a general formula (I) as defined herein, but stabilized with synthetic alpha-tocopherol, an antioxidant disclosed and claimed in U.S. Pat. No. 6,956,062, is tested. This sample is obtained commercially from Schulke GmbH. The tests for all samples determine the formaldehyde and peroxide contents of the test sample when stored at room temperature (RT), for periods of 0, 2, 4, 6, 8, 10 and 12 months. The 0 month sample is the freshly prepared composition.

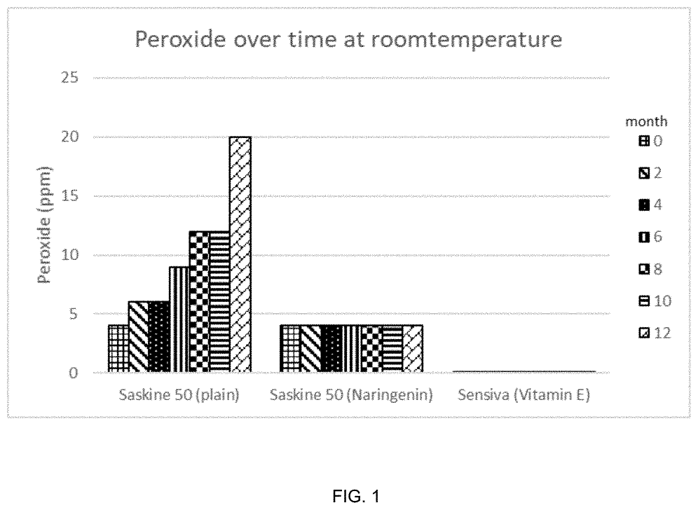

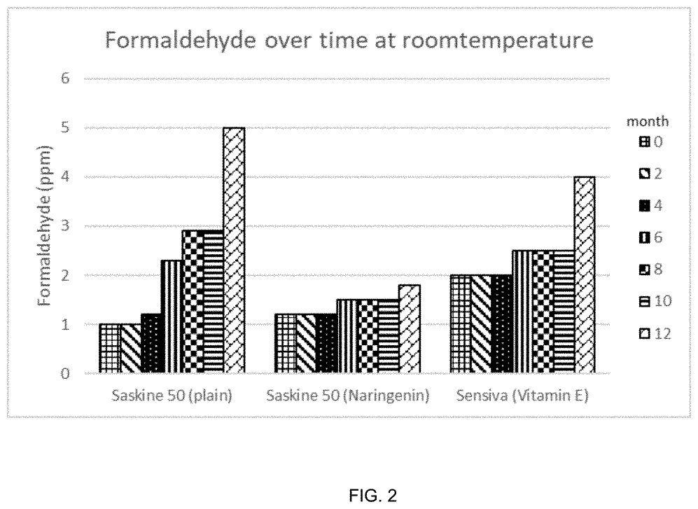

The table below shows the components of the mixtures, and the graphs in FIGS. 1 and 2 show the results of the tests, for formaldehyde and peroxide formation, respectively.

| COMPOUND OF | | | ANTIOXIDANT |
|---|---|---|---|
| FORMULA (I) | SOURCE | ANTIOXIDANT | AMOUNT |
| EHOPD | SACHEM | None | None |
| SASKINE ™ 50 | | | |
| EHOPD | SACHEM | Narigenin | 500 ppm |
| SASKINE ™ 50 | | | |
| EHOPD | SACHEM | Narigenin | 1000 ppm |
| SASKINE ™ 50 | | | |
| EHOPD | SCHULKE | Synthetic alpha- | 500 ppm |
| SENSIVA ® | | tocopherol | |

As evident from FIGS. 1 and 2, the naringenin-containing EHOPD, in accordance with the present invention, provides excellent antioxidant performance compared to both no antioxidant and the synthetic alpha-tocopherol antioxidant of the prior art. Applicant considers that naringenin is representative of the entire class of flavonoids disclosed and claimed in the present application.

### Example 2

A further set of examples further demonstrates the efficacy of the present invention in providing antioxidant activity for 3-[(2-ethylhexyl)oxy]-1,2-propanediol, a compound having a general formula (I) as defined herein, which is marketed under the trade name SASKINE50™ by SACHEM, Inc., Austin, Tex. In this example, SACHEM's SASKINE50™ is used as basis for the stability testing of glycerol alkyl ethers within the scope of the present invention. A pre-defined blend containing SASKINE50™ and a number of the anti-oxidants compounds in accordance with the present invention are prepared, each containing 725 ppm of the antioxidant compound in 99.6% pure SASKINE50™ (3-[(2-ethylhexyl)oxy]-1,2-propanediol). The blend is analyzed via gas chromatography (GC) prior to stability testing. Following the analysis result, the blend is placed under atmospheric pressure and under an inert gas into individual sample bottles as not to disturb the testing conditions during sampling. The test conditions are 50° C. for one month. During the month, the prepared samples are re-analyzed via gas chromatography at intervals of two weeks, for the SASKINE50™ (3-[(2-ethylhexyl)oxy]-1,2-propanediol) assay (loss of the compound) and for formation of volatile by-product. These test conditions are considered to simulate a shelf-life of approximately one year at room temperature.

Both the SASKINE50™ (3-[(2-ethylhexyl)oxy]-1,2-propanediol) and the volatile breakdown products are analyzed by GC. Breakdown products measured are those eluting prior to the SASKINE50™ (3-[(2-ethylhexyl)oxy]-1,2-propanediol). The samples are dissolved in isopropyl alcohol at a concentration of 10% for the GC analysis. The following GC conditions are used:

Column:
Material: fused silica WCOT
Length: 30 m
Internal diameter: 0.32 mm
Stationary phase: DB1701
Film thickness: 1 μm
Gas regulation: Carrier gas: hydrogen
   Make-up gas: helium
Temperatures: Detector: 300° C.
   Injector: 275° C.
   Start temperature: 115° C.
   Warm up speed 1: 8° C./minute
   End temperature: 210° C.
   Warm up speed 2: 15° C./minute
   End temperature: 275° C.

Detection: Type detector: FID

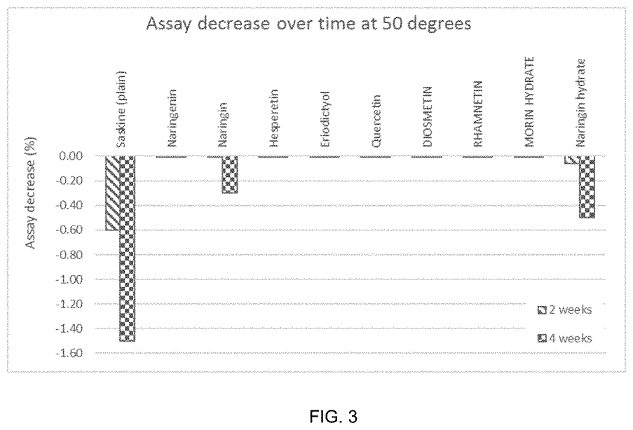

FIG. 3 is a graph showing the loss of SASKINE50™ (3-[(2-ethylhexyl)oxy]-1,2-propanediol) on storage at the elevated temperature of 50° C. with the following compounds in accordance with the present invention: naringenin, naringin, hesperitin, eriodictyol, quercetin, diosmetin, rhamnetin, norinhydrate and naringin hydrate. As shown in the graph in FIG. 3, when no antioxidant is included with the SASKINE50™ (3-[(2-ethylhexyl)oxy]-1,2-propanediol), a significant loss of shown during the test period, whereas when the antioxidant compounds in accordance with the present invention are added, there is almost no loss with all but two of the antioxidant compounds. It is noted that the antioxidant compounds naringin and naringin hydrate performed less well than did the other antioxidant compounds, so that these may be less favored in the present invention.

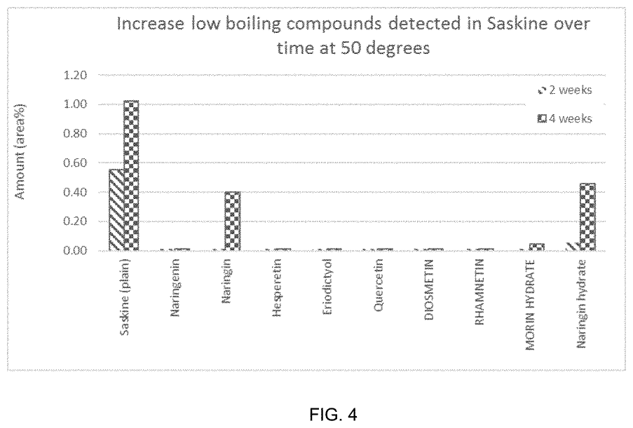

FIG. 4 is a graph showing the increase in volatile degradation products formed on storage of SASKINE50™ (3-[(2-ethylhexyl)oxy]-1,2-propanediol) at the elevated temperature of 50° C. with the same antioxidant compounds in accordance with the present invention as in the test shown in FIG. 3. It is noted that the low boiling compounds are reported as the sum of all detectable compounds which have a lower boiling point, i.e., that elute from the GC column prior to, compared to SASKINE50™ (3-[(2-ethylhexyl)oxy]-1,2-propanediol). As shown in FIG. 4, when no antioxidant is added to the SASKINE50™ (3-[(2-ethylhexyl)oxy]-1,2-propanediol), a significant amount of breakdown products are observed after both 2 weeks and 4 weeks at 50° C. By contrast, significantly smaller amounts of breakdown products are found when using the antioxidants according to the present invention. When the antioxidant compounds in accordance with the present invention are added, there are almost no breakdown products formed with all but two of the antioxidant compounds. It is noted that the antioxidant compounds naringin and naringin hydrate performed less well than did the other antioxidant compounds, so that these may be less favored in the present invention.

### Example 3

A further set of examples further demonstrates the efficacy of the present invention in providing antioxidant activity for 3-[(n-octyl)oxy]-1,2-propanediol, a compound having a general formula (I) as defined herein, which is marketed under the trade name SASKINE80™ by SACHEM, Inc., Austin, Tex. In this example, SACHEM's SASKINE80™ is used as basis for the stability testing of one glycerol alkyl ether within the scope of the present invention, i.e., naringenin. A pre-defined blend containing SASKINE80™ and this anti-oxidant compound in accordance with the present invention are prepared, the blend containing 725 ppm of the naringenin in 99.6% pure SASKINE80™ (3-[(n-octyl)oxy]-1,2-propanediol). The blend is analyzed via gas chromatography (GC) prior to stability testing. Following the analysis result, the blend is placed under atmospheric pressure and under an inert gas into individual sample bottles as not to disturb the testing conditions during sampling. The test conditions are 50° C. for two weeks and four weeks. During this time, the prepared samples are re-analyzed via gas chromatography at intervals of two weeks, for the SASKINE80™ (3-[(n-octyl)oxy]-1,2-propanediol) assay (loss of the compound) and for formation of volatile by-product. These test conditions are considered to simulate a shelf-life of approximately one year at room temperature.

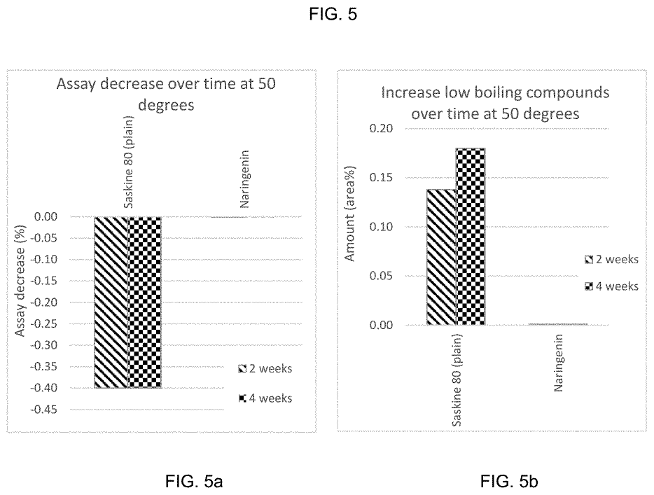

Both the SASKINE80™ (3-[(n-octyl)oxy]-1,2-propanediol) and the volatile breakdown products are analyzed by GC, as described above for Example 2. FIG. 5 shows the results of stability testing of this blend. FIG. 5a shows the SASKINE80™ assay decrease over the two week and four week times at 50° C. for SASKINE80™ alone and the blend of SASKINE80™ with naringenin. As illustrated in FIG. 5a, without the antioxidant, the SASKINE80™ assay decrease is about 0.40%, while with the naringenin, there is little or no assay loss for the SASKINE80™. FIG. 5b shows the increase in low boiling compounds over time at 50° C. for SASKINE80™ alone and the blend of SASKINE80™ with naringenin. As illustrated in FIG. 5b, without the antioxidant, a notable increase in formation of low boiling compounds is observed after both two and four weeks, while with the antioxidant present, there is little or no increase in low boiling compounds over the same time periods at 50° C.

### Example 4

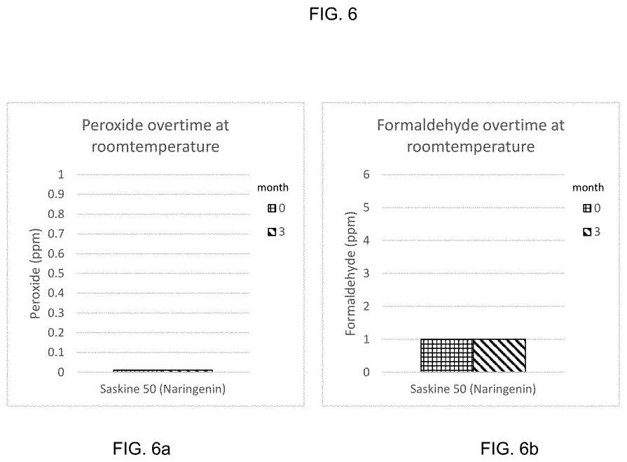

As shown in FIGS. 1 and 2, in the tests in Example 1, even at zero time, there is a measurable, non-zero amount of both peroxide and formaldehyde present in the SASKINE50™. In Example 1, the antioxidant was added subsequent to the manufacture of the SASKINE50™, raising the question of whether formation of the small amounts of peroxide and formaldehyde could be avoided by addition of the antioxidant immediately upon manufacture of the SASKINE50™. Thus, for this Example 4, samples of SASKINE50™ are combined with the antioxidant naringenin immediately upon manufacture, and were tested then (zero time) and three months later. The results are shown in FIG. 6. As shown in FIG. 6a, when the antioxidant is added immediately upon manufacture, the formation of peroxide is inhibited at both zero time and at three months, when stored at room temperature. As shown in FIG. 6b, the same amount of formaldehyde is present at zero time, as that shown at zero time in FIG. 2. However, as shown in FIG. 6b, at three months' time at room temperature, the formaldehyde content has not increased in this Example 4, while in the example shown in FIG. 2, there was an increase in formaldehyde for the same mixture of SASKINE50™ and naringenin when the naringenin is added subsequent to the manufacture of the SASKINE50™. Thus, it is demonstrated that addition of the antioxidant at the time the SASKINE50™ is manufactured has a significant benefit.

### Example 5

As discussed in the background, the purpose of the glyceryl ether compounds such as SASKINE50™ and SASKINE80™ is as a preservative and antimicrobial, especially in cosmetics and pharmaceutical preparations. To test the SASKINE50™ and SASKINE80™, minimum inhibitory concentrations are determined for SASKINE50™ alone, SASKINE50™ with naringenin, SASKINE80™ alone, and SASKINE80™ with naringenin, and, for comparison, Sensiva® SC 50, which contains 3-[(2-ethylhexyl)oxy]-1,2-propanediol and synthetic alpha-tocopherol, which is commercially available from Schülke & Mayr Benelux B.V., 2032 HA-Haarlem, Netherlands. The determination of the minimal inhibitory concentration (MIC) is carried out according to the standard procedure described below.

The purpose of the MIC study is to find a minimum concentration of the test item which will enable to inhibit in vitro the growth of a microbial strain. The MIC characterizes the bacteriostatic or fungistatic effect of a product.

In the MIC test, the product is diluted according to a geometric progression of reason 2 over a range of at least 5 dilutions starting from the maximum concentration to be tested specified by the customer. In each tube of the dilution range, a cultured medium double concentered is included with the strains to be tested. The density of the strains tested are around 106 CFU/ml for bacteria, 105 CFU/ml for yeasts and 104 CFU/ml for molds. Culture medium is Mueller Hinton for bacteria, and Sabouraud for yeast and molds. The incubation for inoculated tubes is at 32.5° C.±2.5° C. for 18-24 h for bacteria, and at 30° C.±2.5° C. for 48 h for yeasts and molds. The tubes showing turbidity related to the growth of the microorganisms are noted. The tubes having no turbidity are then transferred in order to count the remaining microorganisms. The seeded plates are incubated 3 to 5 days in the above-mentioned conditions respectively for bacteria and yeast and molds. Colonies counting on plates is used to calculate the number of CFU (Colony Forming Units) per gram or per ml of product. The MIC corresponds to the concentration of the first tube that does not show microbial turbidity. The CMB corresponds to the concentration of the first tube for which a decrease of 99.99% (4 Log reduction) is obtained for bacteria and of 99.90% (3 Log reduction) for yeasts and molds.

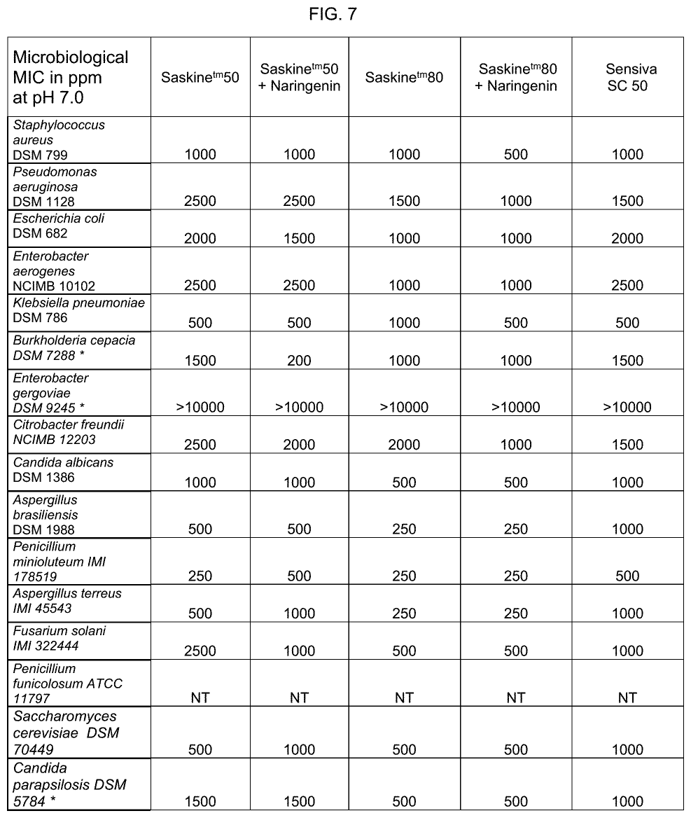

The results are shown in the table in FIG. 7. As shown in FIG. 7, in some cases addition of the stabilizer in ethylhexylglyceryl ether (SASKINE50™) to be more effective in anti-microbial growth compared both to plain material and Sensiva®. As shown in FIG. 7, the n-octyl glyceryl ether (SASKINE80™) shows an even better performance.

### Example 6

As noted herein, the inventive compositions are intended for use in cosmetic and pharmaceutical compositions. One important criteria for such products is that there be no skin irritation, when the compositions are applied to the skin of the person using the cosmetic or pharmaceutical composition. To test the compositions of the present invention, a non-animal test is conducted, which is an in vitro skin irritation study of the compositions on human reconstructed epidermis known as the SkinEthic model, OECD 439. The objective of the study is to evaluate the ability of a product to cause cutaneous irritation by a cytotoxicity test combined with measuring Interleukin 1-alpha (IL1-α) concentrations on reconstructed human epidermis in vitro. After the pure product has been applied to the epidermis for 42 minutes and they have been incubated for 42 hours post-treatment, cell viability is determined by measuring mitochondrial succinate dehydrogenase activity in living cells. This enzyme converts MTT (3(4.5-dimethylthiazol-2-yl)-2,5-diphenyltetrazolium bromide) into formazan blue crystals. A spectrophotometer is used to read the results after the crystals have been dissolved. The measured absorbances are proportional to the number of living cells. In addition, release of inflammation (IL1-α) mediators is measured by colorimeter if cell viability is over 50%. This trial is conducted according to the protocol proposed by ECVAM on Apr. 27 2007 and the OECD 439. Cytotoxicity: absorbance is measured three times at 540 nm. The results are expressed as a viability percentage compared to a negative control:

% viability=Sample absorption×100/Negative control absorption

IL1-α titration: IL1-α concentrations in the culture media are evaluated using the instructions on the titration kit and are expressed as pg/ml.

The test product is considered to be irritant for skin:
if viability after a 42 minutes treatment and 42 hours incubation is less than 50%,
if viability after a 42 minutes treatment and 42 hours incubation is greater than 50%, and the concentration of IL1-α is greater than 50 pg/ml.

The test product is considered to be non irritant for skin:
if viability after a 42 minutes treatment and 42 hours incubation is greater than 50%, and the concentration of IL1-α released is less than or equal to 50 pg/ml.

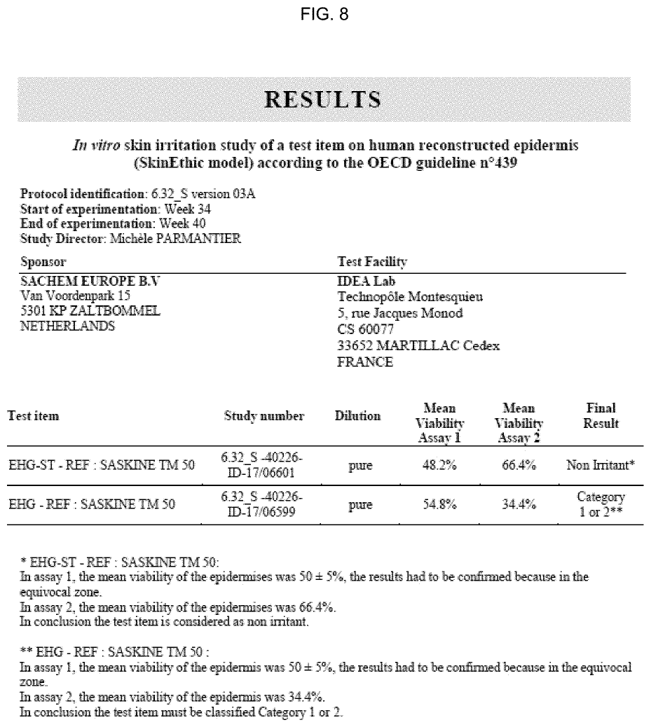

The results of the skin irritation test are shown in FIG. 8. As shown in FIG. 8, the skin irritation tests show that adding naringenin to SASKINE50™, in accordance with the present invention, changes the classification in this test from an irritant of category 1 or 2, to classification as a non-irritant. This is a key factor showing that the present invention provides an unexpected result when used as described herein and tested according to this protocol.

An advantage of the present invention obtains from the fact that naringenin is isolated from citrus sources, such as orange and grapefruit peel, making it easy to trace back its origin. Compositions in accordance with embodiments of the present invention may provide one or more of the following benefits: toxicologically acceptable; readily tolerated by the skin when applied topically; stable; largely and preferably completely odorless; inexpensive to prepare; easy to formulate and not detrimental to final products.

In one embodiment, the present invention provides compositions which comprise one or more glycerol monoalkyl ethers (compound of general formula (I)) and the one or more compound of general formula (II), which are storage-stable for a long period under practical conditions, e.g., when stored at room temperature. In one embodiment, the composition is storage-stable up to 60 months, and in another embodiment, for a period ranging from 12 to 36 months. The compositions in accordance with the present invention should be protected from decomposition, in particular, should be protected from the development of high peroxide numbers, when the composition is tested according to testing procedures standard in the cosmetics and/or pharmaceutical industries.

In summary, the invention provides one or more of the following numbered features.

Feature 1. A composition comprising:

a. a glycerol alkyl ether of the general formula (I):

R—O—CH₂—CHOH—CH₂OH (I)

wherein, in general formula (I), R is a C₃-C₁₈ alkyl or alkenyl group, in which the alkyl or alkenyl group is branched or unbranched, unsubstituted or substituted by one or more hydroxyl, one or more C₁-C₄ alkoxy groups, or both one or more hydroxyl and one or more C₁-C₄ alkoxy groups, and the C₃-C₁₈ alkyl group, whether branched or unbranched, unsubstituted or substituted, is optionally interrupted by up to four oxygen atoms, or R is a C₆-C₁₀ aromatic hydrocarbon, unsubstituted or substituted by one or more hydroxyl, one or more C₁-C₄ alkoxy groups, or both one or more hydroxyl and one or more C₁-C₄ alkoxy groups, and

b. one or more compound selected from general formula (II):

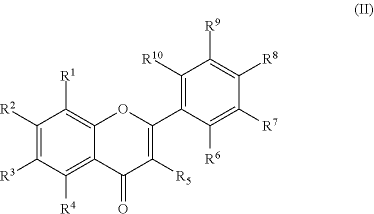

wherein, in the general formula (II), each of R¹-R¹⁰ is independently selected from hydrogen, hydroxyl, alkyl hydroxyl, alkoxy, alkyl ether, alkyl ester and glycoside, wherein the alkyl, the alkoxy and the alkyl portion of the alkyl ester contains from 1 to 4 carbon atoms, branched or unbranched, and the ester of the alkyl ester contains from 1 to 5 carbon atoms, branched or unbranched.
Feature 2. The composition of feature 1 wherein, in the general formula (I), R is a branched or unbranched C₄-C₁₂ alkyl group.
Feature 3. The composition of feature 1 wherein, in the general formula (I), R is 2-ethylhexyl.
Feature 4. The composition of feature 1 wherein, in the general formula (I), R is n-octyl.
Feature 5. The composition of feature 1 wherein, in the general formula (I), R is 2-octyl.
Feature 6. The composition of any one or more of the foregoing features 1-5 wherein, in the one or more compound selected from the general formula (II), each of R¹-R¹⁰ is independently selected from hydrogen, hydroxyl, alkyl hydroxyl and alkoxy.
Feature 7. The composition of any one or more of the foregoing features 1-5 wherein, in the general formula (I), R is a branched or unbranched C₄-C₁₂ alkyl group and in the one or more compound selected from the general formula (II), each of R¹-R¹⁰ is independently selected from hydrogen, hydroxyl, C₁-C₄ alkyl hydroxyl and C₁-C₄ alkoxy.
Feature 8. The composition of any one or more of the foregoing features 1-7 wherein the one or more compound selected from the general formula (II) is one or a combination of two or more of hesperetin, naringenin, taxifolin, epicatechin, isokuranetin, eriodictyol, aromadendrin, aromadedrin, acacetin, isocutellarein, luteolin, kaempferol, quercetin, apigenin, diosmetin, chrysoeriol, chrysin, and galangin.
Feature 9. The composition of feature 1 wherein either R is 2-ethylhexyl or R is n-octyl or R is 2-octyl, and the one or more compound selected from the general formula (II), is one or a combination of two or more of hesperetin, naringenin, taxifolin, epicatechin, isokuranetin, eriodictyol, aromadendrin, aromadedrin, acacetin, isocutellarein, luteolin, kaempferol, quercetin, apigenin, diosmetin, chrysoeriol, chrysin, and galangin.
Feature 10. The composition of feature 1 wherein, in the general formula (I), R is a branched or unbranched C₄-C₁₂ alkyl group and in the one or more compound selected from the general formula (II), R¹, R³, R⁶, R⁷ and R¹⁰ are H, R² and R⁴ are OH, R⁵ and R⁹ are H or OH, and Fe is OH or C₁-C₄ alkoxy.
Feature 11. The composition of any one or more of the foregoing features 1-10 wherein the compound of general formula (II) comprises a flavanone, a flavonol, a flavone, a polymethoxylated flavanone, a polymethoxylated flavonol, a polymethoxylated flavone, a glycoside of a flavanone, a glycoside of a flavonol, a glycoside of a flavone, or a mixture of any two or more of the foregoing.
Feature 12. The composition of any one or more of the foregoing features 1-10 wherein the compound of general formula (II) comprises one or a mixture of two or more of compounds having the general formulae (IIa), (IIb), or (IIc):

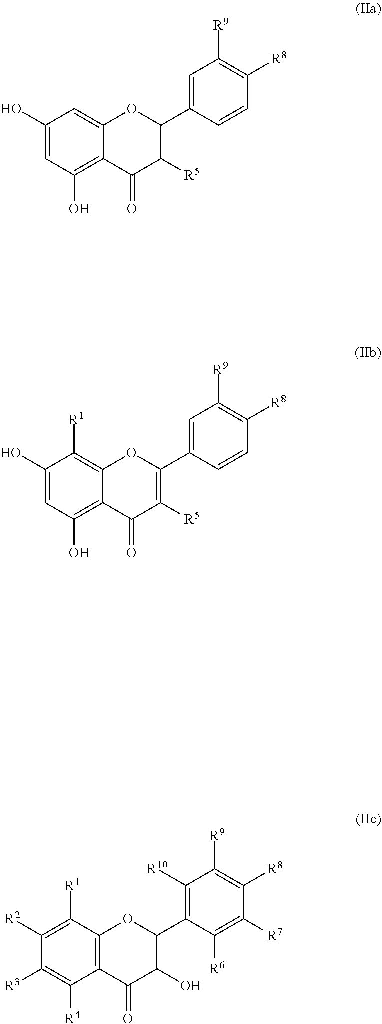

wherein, in the general formulae (IIa), (IIb), and (IIc), each of R¹-R¹⁰ are as defined above for the compound of general formula (II).
Feature 13. The composition of any one or more of the foregoing features 1-12 wherein the composition contains from about 50 ppm to about 50000 ppm of the compound of general formula (II), based on the content of the compound of general formula (I).
Feature 14. The composition of any one or more of the foregoing features 1-13 wherein the composition is a concentrate containing from 0.005 wt % to 5 wt % of the of the compound of general formula (II), based on the total weight of the composition.
Feature 15. The composition of any one or more of the foregoing features 1-14 wherein the compound of general formula (I) is provided to the composition at a purity of at least 95%, prior to addition of the compound of general formula (II).
Feature 16. A cosmetic or pharmaceutical composition comprising the composition of any of any of the foregoing features 1-15.
Feature 17. A cosmetic or pharmaceutical composition comprising the composition of any of any of the foregoing features 1-15, wherein the cosmetic or pharmaceutical composition contains from about 0.05 wt % to about 0.5 wt % of the compound of general formula (I) and from about 50 ppm to about 50000 ppm of the compound of general formula (II) based on the content of the compound of general formula (I) in the cosmetic or pharmaceutical composition.
Feature 18. The composition of any of the foregoing features, comprising:

(a) 60 wt % or less of the compound of general formula (I); and

(b) about 0.05 wt % to about 0.5 wt % of the compound of general formula (II).

Feature 19. The composition of any preceding claim further comprising one or more additive selected from water, ethanol, propylene glycol.

It should be appreciated that the process steps and compositions described herein may not form a complete system or process flow for formulating a cosmetic or pharmaceutical formulation containing the compounds disclosed in the foregoing, such as would be used in actual practice. The present invention can be practiced in conjunction with synthetic organic, formulation and compounding techniques and apparatus currently used in the art, and only so much of the commonly practiced materials, apparatus and process steps are included as are necessary for an understanding of the present invention.

While the principles of the invention have been explained in relation to certain particular embodiments, and are provided for purposes of illustration, it is to be understood that various modifications thereof will become apparent to those skilled in the art upon reading the specification. Therefore, it is to be understood that the invention disclosed herein is intended to cover such modifications as fall within the scope of the appended claims. The scope of the invention is limited only by the scope of the claims.

1. A method of protecting glycerol alkyl ethers from oxidation, comprising step (a.) and step (b.):
a. providing a glycerol alkyl ether of the general formula (Ia) or (Ib):
R—O—CH₂—CHOH—CH₂OH (Ia) or
H—O—CH₂—CHOR—CH₂OH (Ib)
wherein, in general formula (Ia) or (Ib), R is a C₃-C₁₈ alkyl or alkenyl group, in which the alkyl or alkenyl group is branched or unbranched, unsubstituted or substituted by one or more hydroxyl, one or more C₁-C₄ alkoxy groups, or both one or more hydroxyl and one or more C₁-C₄ alkoxy groups, and the C₃-C₁₈ alkyl group, whether branched or unbranched, unsubstituted or substituted, is optionally interrupted by up to four oxygen atoms, or R is a C₆-C₁₀ aromatic hydrocarbon, unsubstituted or substituted by one or more hydroxyl, one or more C₁-C₄ alkoxy groups, or both one or more hydroxyl and one or more C₁-C₄ alkoxy groups, and
b. adding to the glycerol alkyl ether of the general formula (Ia) or (Ib) one or more compound selected from general formula (II): 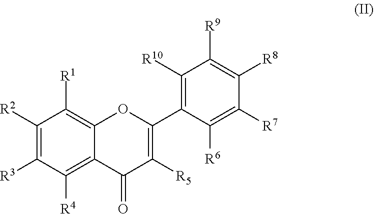
wherein, in the general formula (II), each of R¹-R¹⁰ is independently selected from hydrogen, hydroxyl, alkyl hydroxyl, alkoxy, alkyl ether, alkyl ester and glycoside, wherein the alkyl, the alkoxy and the alkyl portion of the alkyl ester contains from 1 to 4 carbon atoms, branched or unbranched, and the ester of the alkyl ester contains from 1 to 5 carbon atoms, branched or unbranched.

2. The method of claim 1 wherein, in the general formula (Ia) or (Ib), R is a branched or unbranched C₄-C₁₂ alkyl group.

3. The method of claim 1 wherein, in the general formula (Ia) or (Ib), R is 2-ethylhexyl.

4. The method of claim 1 wherein, in the general formula (Ia) or (Ib), R is n-octyl.

5. The method of claim 1 wherein, in the general formula (Ia) or (Ib), R is 2-octyl.

6. The method of claim 1 wherein, in the general formula (Ia) or (Ib), R is heptyl.

7. The method of claim 1 wherein, in the general formula (Ia) or (Ib), R is isoamyl.

8. The method of claim 1 wherein, in the one or more compound selected from the general formula (II), each of R¹-R¹⁰ is independently selected from hydrogen, hydroxyl, alkyl hydroxyl and alkoxy.

9. The method of claim 1 wherein, in the general formula (Ia) or (Ib), R is a branched or unbranched C₄-C₁₂ alkyl group and in the one or more compound selected from the general formula (II), each of R¹-R¹⁰ is independently selected from hydrogen, hydroxyl, C₁-C₄ alkyl hydroxyl and C₁-C₄ alkoxy.

10. The method of claim 1 wherein the one or more compound selected from the general formula (II) is one or a combination of two or more of hesperetin, naringenin, taxifolin, epicatechin, isokuranetin, eriodictyol, aromadendrin, aromadedrin, acacetin, isocutellarein, luteolin, kaempferol, quercetin, apigenin, diosmetin, chrysoeriol, chrysin, and galangin.

11. The method of claim 1 wherein in the general formula (Ia) or (Ib), either R is 2-ethylhexyl or R is n-octyl or R is 2-octyl, and the one or more compound selected from the general formula (II), is one or a combination of two or more of hesperetin, naringenin, eriodyctiol, taxifolin, epicatechin, isokuranetin, aromadendrin, aromadedrin, acacetin, isocutellarein, luteolin, kaempferol, quercetin, apigenin, diosmetin, chrysoeriol, chrysin, and galangin.

12. The method of claim 1 wherein, in the general formula (Ia) or (Ib), R is a branched or unbranched C₄-C₁₂ alkyl group and in the one or more compound selected from the general formula (II), R¹, R³, R⁶, R⁷ and R¹⁰ are H, R² and R⁴ are OH, R⁵ and R⁹ are H or OH, and Fe is OH or C₁-C₄ alkoxy.

13. The method of claim 1 wherein the compound of general formula (II) comprises a flavanone, a flavonol, a flavone, a polymethoxylated flavanone, a polymethoxylated flavonol, a polymethoxylated flavone, a glycoside of a flavanone, a glycoside of a flavonol, a glycoside of a flavone, or a mixture of any two or more of the foregoing.

14. The method of claim 1 wherein the compound of general formula (II) comprises one or a mixture of two or more of compounds having the general formulae (IIa), (IIb), or (IIc): 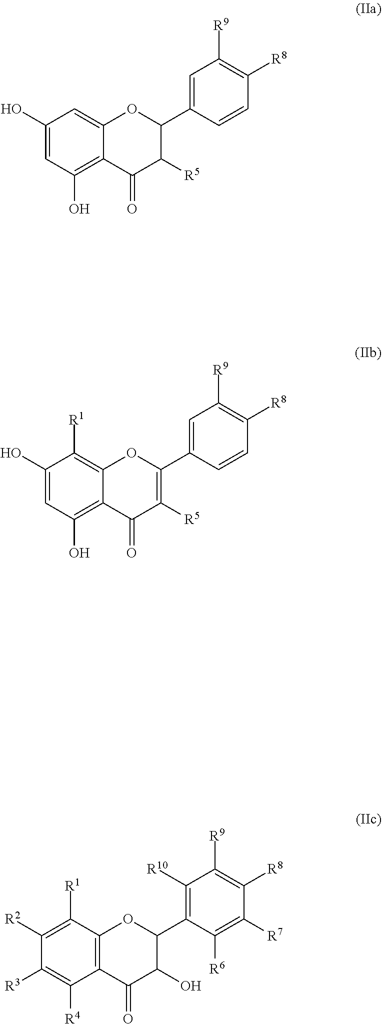
wherein, in the general formulae (IIa), (IIb), and (IIc), each of R¹-R¹⁰ are as defined above for the compound of general formula (II).

15. The method of claim 1 wherein the composition contains from about 50 ppm to about 50000 ppm of the compound of general formula (II), based on the content of the compound of general formula (Ia) or (Ib).

16. The method of claim 1 wherein the compound of general formula (Ia) or (Ib) is provided at a purity of at least 95%, prior to addition of the compound of general formula (II).
